(19) United States
(12) Patent Application Publication
(10) Pub. No.: US 2025/0040904 A1
(43) Pub. Date: Feb. 6, 2025

(54) Parameter Optimization Method and Apparatus, Medical Device, Medium and Product

(71) Applicant: Siemens Healthineers AG, Forchheim (DE)

(72) Inventors: Li Qin Huo, Shenzhen (CN); Jian Qiao Zhang, Shenzhen (CN)

(73) Assignee: Siemens Healthineers AG, Forchheim (DE)

(21) Appl. No.: 18/786,728

(22) Filed: Jul. 29, 2024

(30) Foreign Application Priority Data
Aug. 1, 2023 (CN) 202310963661.3

(51) Int. Cl. A61B 6/00; A61B 6/40; G06T 7/00; G06T 17/00

(52) U.S. Cl. CPC A61B 6/542; A61B 6/4014; A61B 6/482; G06T 7/0014; G06T 17/00

(57) ABSTRACT

The present disclosure relates to parameter optimization. A parameter optimization method may include acquiring an initial exposure parameter usable by the medical device for performing exposure; acquiring contour information of a test subject and initial relative position information of the test subject relative to the examination component; determining an estimated attenuation amount of rays passing through the test subject based on the initial relative position information, contour information of the test subject, and real-time position information of the examination component, the estimated attenuation amount representing the degree of attenuation of rays from the radiation source after passing through the test subject, and the real-time position information of the examination component being position information of the examination component acquired in real time; and updating the initial exposure parameter based on the estimated attenuation amount. The method can reduce the number of exposures and improve the operating procedure and efficiency.

### CROSS REFERENCE TO RELATED APPLICATIONS

This patent application claims priority to Chinese Patent Application No. 202310963661.3, filed Aug. 1, 2023, which is incorporated herein by reference in its entirety.

### BACKGROUND

### Technical Field

The present disclosure relates to the technical field of medical equipment, in particular a parameter optimization method and parameter optimization apparatus for a medical device, a medical device, a computer-readable storage medium and a computer program product.

### Related Art

Adjustment of exposure parameters is an important stage of a workflow of a medical device such as a medical X-ray imaging device. This is because exposure parameters will affect the radiation dose and image quality of the medical X-ray imaging device for example. Choosing suitable exposure parameters is not only beneficial for optimizing image quality to facilitate diagnosis, but can also prevent a test subject from coming into contact with an excessive amount of X-ray radiation.

However, in certain situations, the medical device is unable to acquire an amount of attenuation in the test subject in an exposure path, with the result that a preset exposure curve cannot be adjusted to exposure parameters capable of achieving a diagnostic objective. In this case, an operator needs to perform multiple exposures to obtain multiple medical images, and relies on experience to adjust previously set exposure parameters according to the image quality of the multiple medical images actually obtained. This not only increases the number of invalid exposures, causing resource wastage, but also causes the test subject to come into contact with an excessive amount of X-ray radiation.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a flowchart of a parameter optimization method for a medical device according to one or more exemplary embodiments of the present disclosure.

FIGS. 2A and 2B respectively show schematic drawings of virtual system models according to one or more exemplary embodiments of the present disclosure, viewed from different angles.

FIG. 3 is a schematic block diagram of a parameter optimization apparatus for a medical device according to one or more exemplary embodiments of the present disclosure.

FIG. 4 shows an electronic device according to the disclosure adapted to implement the methods described herein.

The exemplary embodiments of the present disclosure will be described with reference to the accompanying drawings. Elements, features and components that are identical, functionally identical and have the same effect are-insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

According to a first aspect of the present disclosure, a parameter optimization method for a medical device is proposed, the medical device that may comprise an examination component, the examination component that may comprise a radiation source and a detector for receiving rays emitted by the radiation source, and the parameter optimization method that may comprise: acquiring an initial exposure parameter to be used by the medical device for performing exposure; acquiring contour information of a test subject and initial relative position information of the test subject relative to the examination component; determining an estimated attenuation amount of rays passing through the test subject at least on the basis of the initial relative position information and contour information of the test subject and real-time position information of the examination component, the estimated attenuation amount being used to represent the degree of attenuation of rays from the radiation source after passing through the test subject, and the real-time position information of the examination component being position information of the examination component acquired in real time; and updating the initial exposure parameter on the basis of the estimated attenuation amount.

According to a second aspect of the present disclosure, a parameter optimization apparatus for a medical device is proposed, the medical device may comprise an examination component, the examination component may comprise a radiation source and a detector for receiving rays emitted by the radiation source, and the parameter optimization apparatus may comprise: a first acquisition module, configured to acquire an initial exposure parameter to be used by the medical device for performing exposure; a second acquisition module, configured to acquire contour information of a test subject and initial relative position information of the test subject relative to the examination component; a determining module, configured to determine an estimated attenuation amount of rays passing through the test subject at least on the basis of the initial relative position information and contour information of the test subject and real-time position information of the examination component, the estimated attenuation amount being used to represent the degree of attenuation of rays from the radiation source after passing through the test subject, and the real-time position information of the examination component being position information of the examination component acquired in real time; and an update module, configured to update the initial exposure parameter on the basis of the estimated attenuation amount.

According to a third aspect of the present disclosure, a medical device is proposed, which may comprise: at least one processor; and a memory in communicative connection with the at least one processor, wherein the memory stores a computer program which, when executed by the at least one processor, realizes the parameter optimization method according to the present disclosure.

According to a fourth aspect of the present disclosure, a non-transitory computer-readable storage medium storing a computer program is proposed, wherein the computer program, when executed by a processor, realizes the parameter optimization method according to the present disclosure.

According to a fifth aspect of the present disclosure, a computer program product is proposed, comprising a computer program, wherein the computer program, when executed by a processor, realizes the parameter optimization method according to the present disclosure.

According to one or more embodiment of the present disclosure, an estimated attenuation amount of the test subject for rays passing therethrough is determined on the basis of contour information of the test subject, an initial relative position thereof relative to an examination component and a real-time position of the examination component, for the purpose of updating an initial exposure parameter. This enables an initial exposure parameter to be determined more accurately according to the actual circumstances of the test subject in the medical device, so as to reduce the number of times invalid image capture is performed by an operator to adjust the exposure parameter and the time taken to do so, thus improving the procedure and efficiency of medical device operation, and furthermore can reduce the probability that the patient will undergo exposure multiple times, so as to prevent the patient from being subjected to an excessive amount of radiation by radioactive rays.

As a type of medical device, an X-ray imaging device uses an X-ray generator to emit X-rays, the X-ray generator may comprise an X-ray tube (such as a tube) and a high-voltage generator. The X-ray tube generally comprises an anode target and a cathode. A filament of the cathode can be energized to produce thermal electrons, and under the driving action of a high voltage between the cathode and the anode, the electrons move at high speed and strike a surface of the anode target, generating X-ray radiation, and the X-rays are emitted through a window. When the X-ray tube operates, the test subject is exposed to X-rays. Using the X-rays, an operator can irradiate any region to be examined, and then generate an image by means of film or an imaging apparatus. The X-rays penetrate a target or human body and attenuate throughout the process. “Dose” is a measure of an amount of relevant radiation at a particular measurement point, in units of Gy (grays) or uGy (micro-grays). “Incident dose” means the dose of radiation reaching a surface of an object (a surface of the test subject). “Exit dose” means the dose of radiation exiting from the object surface (the test subject surface), directly adjacent to the exit surface. “Absorbed dose” means the dose absorbed by the object when the rays have passed through the object (e.g. the test subject), and represents the degree of attenuation of rays passing through the object. “Water value” simulates the object (e.g. the test subject) as a virtual water column, and is used to represent the degree of absorption (i.e. the degree of attenuation) of rays passing through the object, and may be determined on the basis of the thickness or extension of the part of the object that lies in the path of the rays.

Exposure parameters may for example comprise: tube voltage (alternatively called exposure voltage, in units of kV), tube current (alternatively called exposure current, in units of mA), exposure time (alternatively expressed as exposure pulse width, in units of s or ms), and exposure milliampere-seconds (i.e. the product of tube current and exposure time, in units of mAs). “Tube voltage” may refer to a voltage output by a high voltage generator to the X-ray tube, and represents a penetrating capability of X-rays emitted by the X-ray tube. The higher the tube voltage, the greater the kinetic energy acquired by electrons emitted by the X-ray tube cathode, and the stronger the energy (i.e. penetrating capability) of the X-rays generated. “Tube current” may refer to a current output by the high voltage generator to the X-ray tube, and represents the number of X-rays emitted by the X-ray tube. The greater the tube current, the greater the number of electrons emitted by the X-ray tube cathode, and the greater the number of electrons striking the surface of the anode target every second, and thus the more X-rays generated. “Exposure pulse width” is used to describe the pulse width of X-rays emitted by the X-ray tube.

An exposure curve is used to describe an exposure parameter of each exposure point used by the X-ray tube during operation, wherein the exposure point corresponds to an exposure dose of the X-ray tube, the exposure dose being related to the tube voltage, tube current and exposure time of the X-ray tube. The exposure curve may comprise one or more of the following curves: a voltage curve, a current curve, a time curve (or pulse width curve) and a milliampere-seconds curve. Horizontal coordinates of the voltage curve, the current curve, the time curve and the milliampere-seconds curve represent different exposure points, and the vertical coordinates are respectively used for describing an exposure voltage value, current value, time value or milliampere-second value. The exposure curves are generally calibrated before the medical device is dispatched from the factory.

Adjustment of an exposure parameter is an important stage of a workflow of an X-ray imaging device, for example. Choosing a rational and suitable exposure parameter is not only beneficial for optimizing image quality to facilitate diagnosis, but can also prevent a patient from coming into contact with an excessive amount of X-ray radiation. In the related art, dose adjustment according to a preset exposure curve is the main method for helping a medical device (such as an X-ray imaging device) to find a suitable exposure parameter (for example, enabling an image average grayscale value to reach a target value). Specifically, in a process of actual use of a medical device, there are various interventional imaging techniques, various patients with different conditions, and various regions to be tested in patients. In order to obtain optimal image quality in the process of subjecting a patient to image capture, an imaging protocol will generally be preset for a user in the medical imaging system, and the user can manually select this imaging protocol. Multiple imaging protocols are predefined for different imaging regions, patient dimensions and patient postures. These imaging protocols are generally related to operating parameters to be realized by a control apparatus of the medical imaging system, such as X-ray generator frame rate, radiation dose, noise processing and signal post-processing, etc., enabling the user to provide an image capture mode giving the clearest images on the basis of the target region of the patient that currently needs to undergo image capture. Such an imaging protocol, especially in medical imaging systems, is also called an organ program (OGP). In a process of actual use, normally a medical device operator, by means of a human-machine interactive device (such as a touchscreen, keyboard, mouse, etc.), can choose a corresponding organ program (OGP) according to an organ/region to be tested, patient body type and posture (such as upright or lateral); thus, the medical device, on the basis of a preset exposure curve corresponding to the organ program, can automatically determine an expected X-ray dose corresponding to this organ/region, and determine a tube voltage value, tube current value, exposure time length, etc. of the X-ray tube. Thereafter, the X-ray imaging device can generally adjust the exposure parameters by a double-exposure method; for example, dose adjustment is first performed according to a preset exposure curve by means of a single brief pre-exposure to obtain a more suitable exposure parameter, which is then used to perform exposure. Alternatively, the patient may be subjected to exposure multiple times, and based on the quality of images obtained by the multiple exposures, an accurate absorbed dose of the region to be tested is determined, in order to update the exposure parameter.

However, the scenario described above might result in the operator performing re-exposure multiple times and spending more operating time, and will cause the test subject to be subjected to an excessive radiation dose.

In the present disclosure, an estimated attenuation amount of the test subject for rays passing therethrough is determined on the basis of contour information of the test subject, an initial relative position thereof relative to an examination component and a real-time position of the examination component, for the purpose of updating an initial exposure parameter. This enables an initial exposure parameter to be determined more accurately according to the actual circumstances of the test subject in the medical device, so as to reduce the number of times invalid image capture is performed by an operator to adjust the exposure parameter and the time taken to do so, thus improving the procedure and efficiency of medical device operation, and furthermore can reduce the probability that the patient will undergo exposure multiple times, so as to prevent the patient from being subjected to an excessive amount of radiation by radioactive rays.

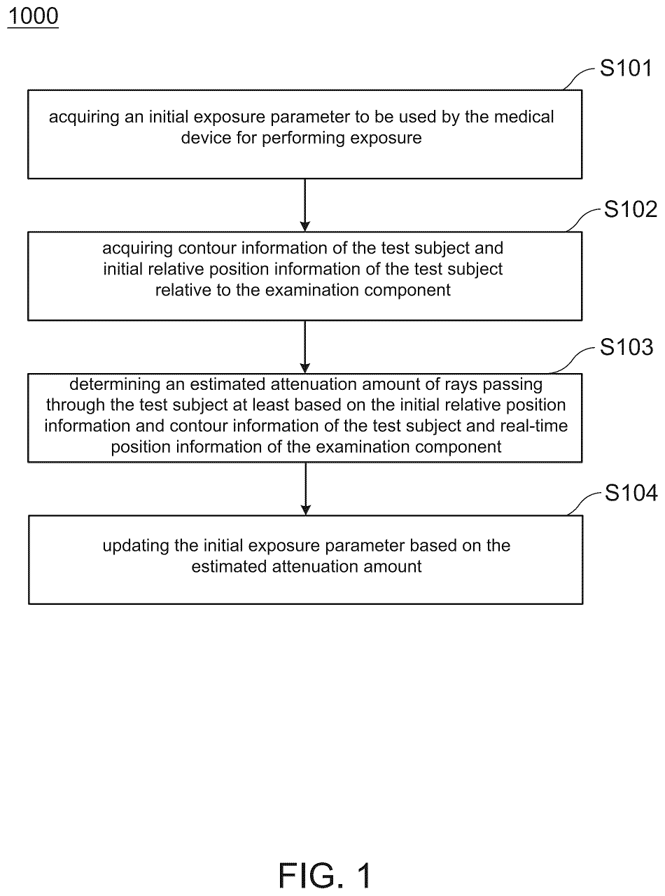

FIG. 1 is a flowchart of a parameter optimization method 1000 for a medical device according to some exemplary embodiments of the present disclosure. The medical device may be an X-ray imaging device (e.g. a C-arm X-ray imaging device), etc. The medical device may comprise an examination component. The examination component may comprise a radiation source (e.g. an X-ray tube), a detector for receiving rays emitted by the radiation source, or an examination table, etc. The test subject may be positioned on the examination table, and rays from the radiation source will pass through the test subject and the examination table to reach a detector, and be received by the detector. The radiation source, detector and examination table can move during actual use, so as to be adjusted to suitable positions so that rays from the radiation source can pass through a target region to be tested of the test subject. As shown in FIG. 1, the parameter optimization method 1000 may comprise:
step S101, acquiring an initial exposure parameter to be used by the medical device for performing exposure;
step S102, acquiring contour information of the test subject and initial relative position information of the test subject relative to the examination component;
step S103, determining an estimated attenuation amount of rays passing through the test subject at least on the basis of the initial relative position information and contour information of the test subject and real-time position information of the examination component; and
step S104, updating the initial exposure parameter on the basis of the estimated attenuation amount.

In step S101, the initial exposure parameter to be used by the medical device for performing exposure is acquired.

Here, “exposure” may refer to any one instance of exposure performed by the medical device. For example, “exposure” may be exposure performed on a patient on the basis of a selected organ program, in which case an exposure parameter determined according to a preset exposure curve selected by the organ program is the initial exposure parameter. As another example, “exposure” may also be a second exposure following a first exposure, in which case an exposure parameter at the moment when the first exposure ends is the initial exposure parameter. In some examples, the initial exposure parameter may comprise one or more of initial exposure voltage, initial exposure current and initial exposure pulse width, etc.

In step S102, the contour information of the test subject and initial relative position information of the test subject relative to the examination component are acquired.

The test subject is for example a human body. The region to be tested of the test subject may be a region of the human body that needs to undergo radiographic examination, such as a hand, leg, chest, etc. For example, an image comprising the examination component and the test subject may be acquired by means of a sensor attached to the radiation source (e.g. a camera such as a 3D camera or depth camera, or a laser scanner) or an additional sensor positioned independently of the radiation source (e.g. arranged on a wall of an examination room), in order to obtain from the image the contour information of the test subject and initial relative position information of the test subject relative to the examination component. Initial relative position information of the test subject relative to the examination component may also be measured by means of another sensor, e.g. a sensor arranged on the examination component. The initial relative position of the test subject relative to the examination component is initially (i.e. when the test subject has lain down at a suitable position on the examination table and no longer moves, i.e. at a fixed position) acquired relative position information of the test subject relative to the examination component at this moment. In some examples, the initial relative position information of the test subject relative to the examination component may be relative position information of the test subject relative to the radiation source and/or the detector (for example, relative position coordinates or another relative positional relationship), or may be relative position information of the test subject relative to the examination table; these relative positional relationships can all reflect the relative positional relationships of the test subject to the various examination components (because the position of each examination component itself will also be acquired).

After obtaining the initial relative position information of the test subject relative to the examination component, relative positions of the test subject and the examination component in a virtual system model (described in detail below) for example can be subsequently updated on the basis of initial position information of the examination component (i.e. corresponding to the initial relative position information of the test subject relative to the examination component) and/or post-movement real-time position information thereof, so that the virtual system model can reflect the actual relative position situation of the test subject and the examination component, to facilitate determination of the estimated attenuation amount.

In step S103, the estimated attenuation amount of rays passing through the test subject is determined at least on the basis of the initial relative position information and contour information of the test subject and real-time position information of the examination component.

The real-time position information of the examination component is position information of the examination component acquired in real time. The examination component can adjust its own position as required; therefore, the medical device needs to acquire position information of the examination component in real time, in order to update the positions of various virtual component models in a virtual system model for example, thereby ensuring that a relative positional relationship between the test subject and examination component in the virtual system model is accurate. The real-time position information of the examination component may be position information of the examination component acquired at the moment when step S103 is performed, or may be position information of the examination component acquired at the same moment that step S102 is performed. In summary, the real-time position information of the examination component can reflect the latest position information (which may have changed from the initial position of the examination component, or may remain the same as the initial position) of the examination component at the moment when step S103 is performed; in this way, it is possible to ensure that the virtual system model for example can reflect the actual relative position situation of the test subject and the examination component, i.e. the relative position situation fixed for performing exposure.

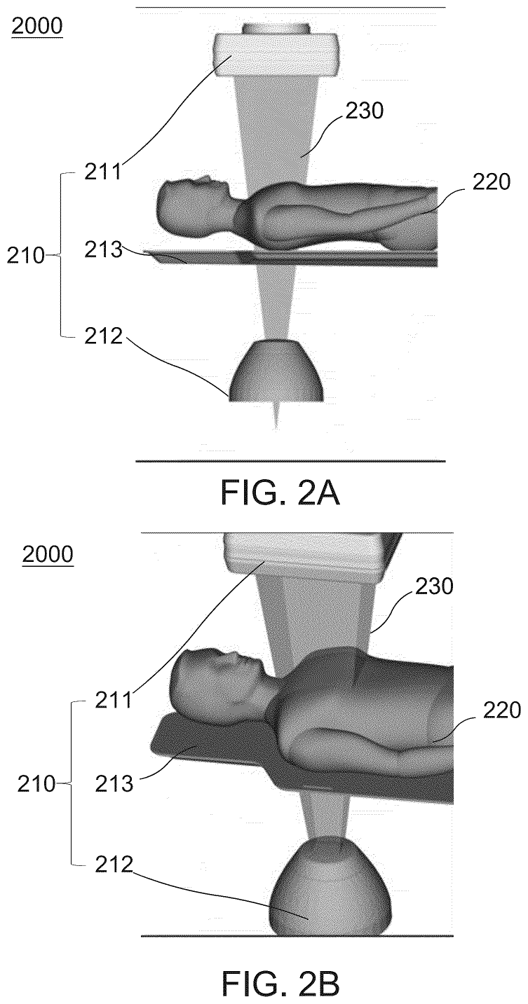

The estimated attenuation amount is used to represent the degree of attenuation of rays from the radiation source after passing through the test subject, and for example is associated with the thickness of the part of the test subject that lies in the ray path (and a ray absorption characteristic of this part), etc. Here, the ray path is the path which the rays must pass along from the radiation source until received by the detector, e.g. the ray path 230 shown in FIGS. 2a and 2b. The estimated attenuation amount may be expressed as an equivalent water value of the part of the test subject that lies in the ray path, or directly as size information (e.g. thickness or volume) or another parameter of the part of the test subject that lies in the ray path. It should be understood here that the estimated attenuation amount may also be another quantity used to represent the degree of attenuation of rays from the radiation source after passing through the test subject. Thus, size information (specifically, thickness) of the part of the test subject that lies in the ray path can be determined by means of the initial relative position information and contour information of the test subject and the real-time position information of the examination component, and this size information serves directly as the estimated attenuation amount or is converted to an equivalent water value to serve as the estimated attenuation amount.

In some examples, based on the initial relative position information and contour information of the test subject and the real-time position information of the examination component, a corresponding mathematical model (such as a formula) or a geometric model (a 2D model or 3D model) may be constructed, and size information of the part of the test subject that lies in the ray path may be determined from the relative positional relationship, reflected by the model, between the region to be tested of the test subject and the ray path. The size information may serve directly as the estimated attenuation amount, or an estimated water value may be determined at least on the basis of the size information.

In step S104, the initial exposure parameter is updated on the basis of the estimated attenuation amount.

The estimated attenuation amount is used to represent the degree of attenuation of rays from the radiation source after passing through the test subject, and may reflect the degree of absorption of rays after passing through the test subject, i.e. is associated with the absorbed dose. Thus, an estimated exposure dose may be determined on the basis of the estimated attenuation amount, the initial exposure parameter then being updated on the basis of the estimated exposure dose, e.g. according to a preset exposure curve.

The embodiment described above enables an initial exposure parameter to be determined more accurately according to the actual circumstances of the test subject in the medical device, so as to reduce the number of times invalid image capture is performed by an operator to adjust the exposure parameter and the time taken to do so, thus improving the procedure and efficiency of medical device operation, and furthermore can reduce the probability that the patient will undergo exposure multiple times, so as to prevent the patient from being subjected to an excessive amount of radiation by radioactive rays. The parameter optimization method of the present disclosure is especially suitable for a digital radiography (DR) process in which only a single exposure is performed. It will be readily understood that the parameter optimization method of the present disclosure is likewise suitable for application scenarios in which continuous exposure must be performed.

In some embodiments, step S102 may comprise: acquiring an initial image of the test subject located in the medical device; and determining contour information of the test subject and initial relative position information thereof relative to the examination component on the basis of the initial image. This facilitates the acquisition of contour information of the test subject and initial relative position information thereof relative to the examination component.

In some embodiments, in the case where the estimated attenuation amount is an estimated water value, etc., step S103 may comprise: determining size information of the part of the test subject that lies in the ray path at least on the basis of the initial relative position information and contour information of the test subject and real-time position information of the examination component; and determining an estimated attenuation amount of the test subject at least on the basis of the size information. As shown in FIGS. 2a and 2b, the ray path 230 is the path which rays must pass along from the radiation source 211 until received by the detector 212. In the embodiment described above, size information (e.g. volume/thickness) of the part of the test subject that lies in the ray path can be determined simply, effectively and accurately on the basis of the position information mentioned above, so as to accurately estimate an exposure dose in order to obtain a suitable exposure parameter. Alternatively, if the estimated attenuation amount is size information, the estimated attenuation amount may be determined directly on the basis of the initial relative position information and contour information of the test subject and real-time position information of the examination component.

In some embodiments, the step of determining size information of the part of the test subject that lies in the ray path at least on the basis of the initial relative position information and contour information of the test subject and real-time position information of the examination component may comprise: determining a virtual system model (e.g. the virtual system model 2000 shown in FIGS. 2a and 2b) of the medical device at least on the basis of the initial relative position information and contour information of the test subject and real-time position information of the examination component; and at least determining size information of the part of the test subject that lies in the ray path on the basis of the virtual system model. As shown in FIGS. 2a and 2b, the virtual system model 2000 may comprise a virtual subject model 220 corresponding to the test subject, and a virtual component model 210 corresponding to the examination component. The virtual component model 210 corresponding to the examination component may comprise a virtual radiation source model 211 corresponding to the radiation source, and a virtual detector model 212 corresponding to the detector. Additionally, the virtual component model 210 corresponding to the examination component may further comprise a virtual examination table model 213 corresponding to the examination table. In some examples, size information of the part of the test subject that is located in the ray path 230 may be determined on the basis of a relative positional relationship, reflected in the virtual system model 2000, between the region to be tested of the test subject and the ray path 230. Alternatively, size information of the part of the test subject that is located in the ray path 230 may be obtained by measurement directly from the virtual system model 2000. As described above, size information of the part of the test subject that is located in the ray path can be determined simply, efficiently and accurately by means of the virtual system model, thereby facilitating the determination of the water value or even the exposure parameter. In some embodiments, the parameter optimization method described above is performed when the position of the test subject is already determined and no longer moves but the position of the examination component might be adjusted or might not be adjusted. In this case, the step of determining a virtual system model of the medical device at least on the basis of the initial relative position information and contour information of the test subject and real-time position information of the examination component, may comprise: constructing a virtual subject model relative to a virtual component model at least on the basis of the initial relative position information and contour information of the test subject, the virtual component model being a virtual model pre-stored in the medical device; and updating the virtual component model on the basis of the real-time position information of the examination component. That is to say, for example, in the case where a virtual component model relating to the examination component is pre-stored in the medical device, the positions of various parts in the pre-stored virtual component model correspond to the initial positions of various components in the examination component; in this case, a virtual subject model corresponding to the contour of the test subject may be constructed on the basis of the contour information of the test subject, and based on the initial relative position of the test subject relative to the examination component, the virtual subject model may be placed in a corresponding position within the virtual component model (a corresponding position relative to one or more component model in the virtual component model). It should be understood here that in order to construct the virtual system model comprising the virtual subject model, the initial relative position information of the test subject relative to the examination component may be relative position information of the test subject relative to at least one of the detector, the radiation source and the examination table; all of these items of initial relative position information enable the virtual subject model to be placed in a position in the virtual component model that corresponds to the actual situation. Next, as shown in FIGS. 2a and 2b, after placing the virtual subject model 213 into the virtual component model 210, the virtual component model 210 can be updated on the basis of real-time position information of the examination component. In this way, it can be ensured that the relative positional relationship between the virtual subject model 213 and the virtual component model 210 in the virtual system model 2000 remains consistent with the actual relative positional relationship between the test subject and the examination component, regardless of whether the examination component moves relative to its initial position.

In particular, the initial relative position information of the test subject relative to the examination component may comprise initial relative position information of the test subject relative to the examination table. Since the test subject is lying on the examination table, the position of the test subject relative to the examination table does not change; the acquisition of the initial relative position information of the test subject relative to the examination table can facilitate positioning of the virtual subject model in the virtual system model, and make it easier to simply and quickly update the virtual system model comprising the virtual subject model when the positions of the detector and the radiation source change.

In some other embodiments, the parameter optimization method described above is performed when the positions of the test subject and the examination component are both already determined and not subsequently adjusted. In this case, the step of determining a virtual system model of the medical device at least on the basis of the initial relative position information and contour information of the test subject and real-time position information of the examination component, may comprise: updating a virtual component model on the basis of real-time position information of the examination component; and constructing a virtual subject model relative to the updated virtual component model at least on the basis of the initial relative position information and contour information of the test subject. That is to say, if the positions of the test subject and the examination component have both been adjusted to suitable positions and are not subsequently adjusted, the relative positional relationship of the test subject to the examination component will also not change, i.e. the real-time position information of the examination component remains the same as initial position information thereof corresponding to the initial relative position information of the test subject. In this case, a pre-stored virtual component model may first be updated on the basis of real-time position information of the examination component, then a virtual subject model may be constructed relative to the updated virtual component model on the basis of the initial relative position information and contour information of the test subject (the virtual subject model constructed on the basis of contour information of the test subject being placed at a corresponding position relative to one or more component model in the virtual component model). In this way, it can still be ensured that the relative positional relationship between the virtual subject model and virtual component model in the virtual system model is consistent with the relative positional relationship between the actual test subject and examination component.

It should be understood here that the virtual subject model could also be a model pre-stored in the medical device, in which case, a contour of the pre-stored virtual subject model may be updated on the basis of contour information of the test subject.

In some embodiments, the step of constructing a virtual subject model relative to a virtual component model at least on the basis of the initial relative position information and contour information of the test subject, may comprise: constructing a virtual subject model relative to a virtual component model on the basis of the initial relative position information and contour information of the test subject and a virtual organ model, the virtual organ model being a virtual model pre-stored in the medical device and used to determine a ray absorption characteristic of the part of the test subject that lies in the ray path (specifically, an internal organ structure), wherein the step of at least determining size information of the part of the test subject that lies in the ray path on the basis of the virtual system model may comprise: determining size information and a ray absorption characteristic of the test subject in the ray path on the basis of the virtual system model, for the purpose of determining an estimated attenuation amount. The embodiment described above is especially suitable for cases where the estimated attenuation amount is a water value; by constructing a virtual subject model on the basis of the contour information of the test subject and a virtual organ model, two factors may be taken into account in the virtual subject model, namely the size of the region to be tested and the ray absorption characteristic of the internal organ structure, such that the equivalent water value of the test subject that is determined is more accurate.

In some embodiments, step S104 may comprise: updating the initial exposure parameter according to an exposure curve on the basis of the estimated attenuation amount. Due to the estimated attenuation amount (which may be converted to an estimated exposure dose), a new exposure point may be determined according to an exposure curve on the basis of the estimated attenuation amount, so as to update the initial exposure parameter.

In some embodiments, the parameter optimization method 1000 may further comprise: causing the medical device to use the updated initial exposure parameter to perform exposure, so as to obtain a medical image; determining an actual attenuation amount of the test subject on the basis of an image parameter of the medical image; and determining whether to update the updated initial exposure parameter again on the basis of the estimated attenuation amount and the actual attenuation amount. The image parameter may be grayscale value, contrast-to-noise ratio (CNR), signal-to-noise ratio (SNR), etc. The step of determining whether to update the updated initial exposure parameter again on the basis of the estimated attenuation amount and the actual attenuation amount may comprise: determining a difference value or ratio of the estimated attenuation amount and actual attenuation amount; in response to the difference value or ratio of the estimated attenuation amount and actual attenuation amount being greater than a preset threshold range, determining to update the updated initial exposure parameter again; and in response to the difference value or ratio of the estimated attenuation amount and actual attenuation amount being less than or equal to a preset threshold range, determining to not update the updated initial exposure parameter again, i.e. continuing to use the updated initial exposure parameter. The embodiment described above enables verification of the estimated attenuation amount. If the estimated attenuation amount and the actual attenuation amount determined according to the estimated attenuation amount are not the same or a permitted tolerance range is exceeded, it can be determined that this estimated attenuation amount might make it impossible for the target dose to be achieved by the medical image obtained by the exposure parameter thereby determined. In this case, it is possible to continue using, for example, a pre-exposure program in the medical device to perform dose adjustment according to the exposure curve.

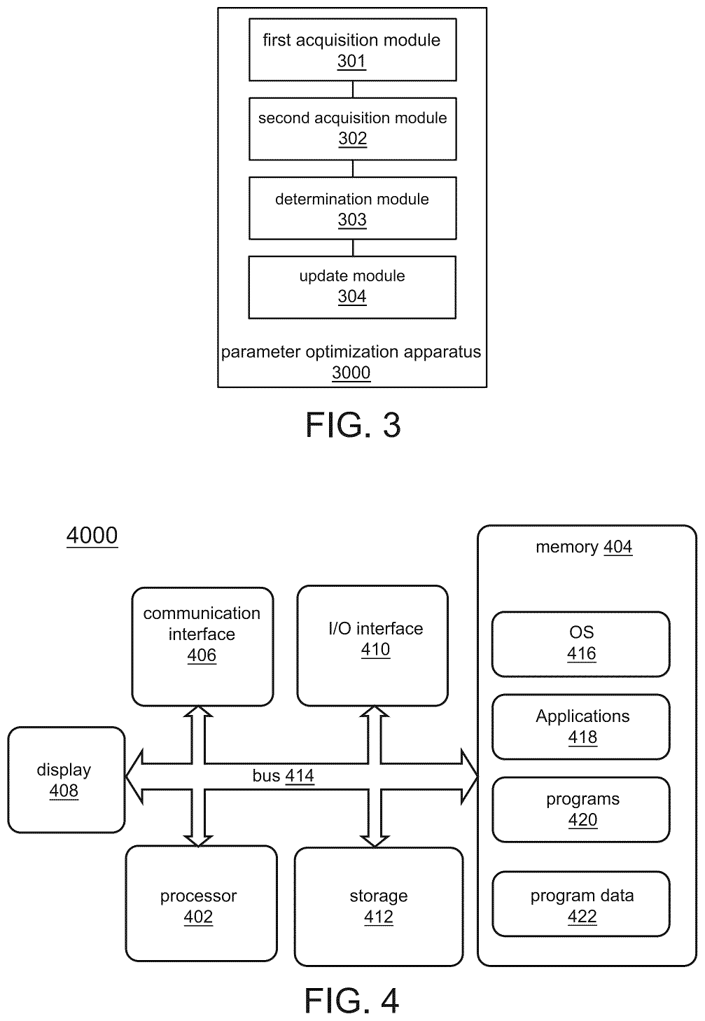

FIG. 3 is a schematic block diagram of a parameter optimization apparatus 3000 for a medical device according to some exemplary embodiments of the present disclosure. As shown in FIG. 3, the parameter optimization apparatus 3000 may comprise: a first acquisition module 301, a second acquisition module 302, a determination module 303 and an update module 304. The first acquisition module (also referred to herein as first acquisition circuitry or first acquisition processor) 301 is configured to acquire an initial exposure parameter to be used by the medical device for performing exposure. The second acquisition module (also referred to herein as second acquisition circuitry or second acquisition processor) 302 is configured to acquire contour information of the test subject and initial relative position information of the test subject relative to the examination component. The determining module (also referred to herein as determination circuitry or determination processor) 303 is configured to determine an estimated attenuation amount of rays passing through the test subject at least on the basis of the initial relative position information and contour information of the test subject and real-time position information of the examination component, the estimated attenuation amount being used to represent the degree of attenuation of rays from the radiation source after passing through the test subject, and the real-time position information of the examination component being position information of the examination component acquired in real time. The update module (also referred to herein as updater, update circuitry, or update processor) 304 is configured to update the initial exposure parameter based on the estimated attenuation amount.

It should be understood that the modules of the apparatus 3000 shown in FIG. 3 may correspond to the steps in the method 1000 described with reference to FIG. 1. Thus, the operations, features and advantages described above for the method 1000 likewise apply to the apparatus 3000 and the modules comprised therein. For conciseness, some operations, features and advantages are not described again here.

According to another aspect of the present disclosure, a medical device is provided, which may comprise: at least one processor; and a memory in communicative connection with the at least one processor, wherein the memory stores a computer program which, when executed by the at least one processor, realizes steps in the method 1000 as described above. The medical device may be an X-ray imaging device, etc.

According to another aspect of the present disclosure, an electronic device is provided, which may comprise: at least one processor; and a memory in communicative connection with the at least one processor, wherein the memory stores a computer program which, when executed by the at least one processor, realizes steps in the method 1000 as described above. The X-ray imaging device comprises this type of electronic device.

According to another aspect of the present disclosure, a non-transitory computer-readable storage medium storing a computer program is provided, wherein the computer program, when executed by a processor, realizes steps in the method 1000 as described above.

According to another aspect of the present disclosure, a computer program product is provided, comprising a computer program which, when executed by a processor, realizes steps in the method 1000 described above.

Illustrative examples of such an electronic device, non-transitory computer-readable storage medium and computer program product are described below with reference to FIG. 4.

FIG. 4 shows an exemplary configuration of an electronic device 4000 that may be used to implement the methods described herein. The electronic device 4000 may be a variety of different types of device, e.g. a server of a service provider, a device associated with a client (e.g. a client device), a system on a chip, and/or any other suitable computer device or computing system. Examples of the electronic device 4000 include but are not limited to: a desktop computer, a server computer, a notebook computer or netbook computer, a mobile device (for example, a tablet computer, cellular or other wireless telephone (e.g. smartphone), notepad computer, mobile station), etc.

The electronic device 4000 may comprise the following, capable of communicating with each other for example by means of a system bus 414 or other suitable connection: at least one processor 402, a memory 404, (multiple) communication interface(s) 406, a display device 408, another input/output (I/O) device 410 and one or more large-capacity storage device 412.

The processor 402 may be a single processing unit or multiple processing units, and all of the processing units may comprise a single or multiple computing units or multiple cores. The processor 402 may be implemented as one or more microprocessor, microcomputer, microcontroller, digital signal processor, central processing unit, state machine, logic circuit and/or any device which controls signals on the basis of operating instructions. Besides other abilities, the processor 402 may be configured to acquire and execute computer-readable instructions stored in the memory 404, large-capacity storage device 412 or other computer-readable medium, such as program code of an operating system 416, program code of an application program 418, program code of another program 420, etc.

The memory 404 and large-capacity storage device 412 are examples of computer-readable storage media used to store instructions, which are executed by the processor 402 to implement the various functions described above. As an example, the memory 404 may generally comprise both a volatile memory and a non-volatile memory (e.g. RAM, ROM, etc.). In addition, the large-capacity storage device 412 may generally comprise a hard disk drive, solid state drive, removable media, including external and removable drives, memory cards, flash memory, floppy disks, optical disks (e.g. CD, DVD), storage arrays, network attached storage, storage area networks, etc. The memory 404 and large-capacity storage device 412 may both be collectively referred to as memory or computer-readable storage media herein, and may be non-transitory media capable of storing computer-readable, processor-executable program instructions as computer program code, which may be executed by the processor 402 as a specific device configured to implement the operations and functions described in the examples herein.

Multiple program modules may be stored on the large-capacity storage device 412. These programs comprise the operating system 416, one or more application program 418, other program 420 and program data 422, and they may be loaded to the memory 404 for execution. Examples of such application programs or program modules may include, for example, computer program logic for realizing the following components/functions (e.g. computer program code or instructions): the apparatus 3000 (including the first acquisition module 301, second acquisition module 302, determining module 303 and update module 304), the method 1000 (including any suitable steps of the method 1000) and/or other embodiments described herein.

Although shown in FIG. 4 as being stored in the memory 404 of the computer device 4000, the modules 416, 418, 420 and 422 or parts thereof may be implemented using any form of computer-readable media that can be accessed by the computer device 4000. As used herein, “computer-readable media” include at least two types of computer-readable media: computer storage media and communication media.

Computer storage media include volatile and non-volatile, removable and non-removable media implemented by any method or technology used to store information such as computer-readable instructions, data structures, program modules or other data. Computer storage media include but are not limited to RAM, ROM, EEPROM, flash memory or other memory technologies, CD-ROM, digital versatile disks (DVD) or other optical storage means, magnetic cartridges, magnetic tape, magnetic disk storage means or other magnetic storage devices, or any other non-transmitting medium that can be used to store information for access by a computer device.

In contrast, communication media may specifically realize computer-readable instructions, data structures, program modules or other data in modulated data signals such as carriers or other transmission mechanisms. As defined herein, computer storage media do not include communication media.

The electronic device 4000 may further comprise one or more communication interface 406 for exchanging data with another device, for example by means of a network, direct connection, etc., as discussed above. Such a communication interface may be one or more of the following: any type of network interface (e.g. a network interface card (NIC)), wired or wireless (e.g. IEEE 802.11 wireless LAN (WLAN)) interface, Worldwide Interoperability for Microwave Access (Wi-MAX) interface, Ethernet interface, universal serial bus (USB) interface, cellular network interface, Bluetooth™ interface, near-field communication (NFC) interface, etc. The communication interface 406 can promote communication in various network and protocol types, including wired networks (e.g. LAN, electric cable, etc.) and wireless networks (e.g. WLAN, cellular, satellite, etc.), internet, etc. The communication interface 406 may also provide communication with an external storage apparatus (not shown) in, for example, a storage array, network attached storage, storage area network, etc.

In some examples, a display device 408 such as a monitor may be included, for displaying information and images to a user. Other I/O devices 410 may be devices that receive various inputs from the user and provide various outputs to the user, and may include a touch input device, gesture input device, camera, keyboard, remote controller, mouse, printer, audio input/output device, etc.

The above are merely embodiments of the present disclosure, which are not intended to limit it. Any amendments, equivalent substitutions or improvements, etc. made within the spirit and principles of the present disclosure shall be included in the scope of protection thereof.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A parameter optimization method for a medical device including an examination component having a radiation source and a detector adapted to receive rays emitted by the radiation source, the parameter optimization method comprising:
acquiring an initial exposure parameter usable by the medical device for performing exposure;
acquiring contour information of a test subject and initial relative position information of the test subject relative to the examination component;
determining, based on the initial relative position information, the contour information, and real-time position information of the examination component, an estimated attenuation amount of rays passing through the test subject, he estimated attenuation amount representing a degree of attenuation of rays from the radiation source after passing through the test subject, and the real-time position information of the examination component being position information of the examination component acquired in real time; and
updating the initial exposure parameter based on the estimated attenuation amount.

2. The parameter optimization method as claimed in claim 1, wherein:
the determining the estimated attenuation amount comprises: determining size information of a part of the test subject lying in a ray path at least based on the initial relative position information, the contour information of the test subject, and the real-time position information of the examination component, the ray path being a path in which rays pass along from the radiation source until received by the detector; and
the estimated attenuation amount of the test subject is determined further based on the size information.

3. The parameter optimization method as claimed in claim 2, wherein determining size information comprises: determining a virtual system model of the medical device at least based on the initial relative position information, the contour information of the test subject, and the real-time position information of the examination component, the virtual system model comprising a virtual subject model corresponding to the test subject and a virtual component model corresponding to the examination component, the size information of the part of the test subject being determined based on the virtual system model.

4. The parameter optimization method as claimed in claim 3, wherein determining the virtual system model of the medical device comprises:
constructing the virtual subject model relative to the virtual component model at least based on the initial relative position information and the contour information of the test subject, the virtual component model being a virtual model pre-stored in the medical device; and
updating the virtual component model based on the real-time position information of the examination component.

5. The parameter optimization method as claimed in claim 4, wherein:
constructing the virtual subject model relative to the virtual component model comprises: constructing the virtual subject model relative to the virtual component model based on the initial relative position information, the contour information of the test subject, and a virtual organ model, the virtual organ model being a virtual model pre-stored in the medical device and useable to determine an absorption characteristic for rays of the part of the test subject that lies in the ray path; and
determining the size information of the part of the test subject comprises: determining size information and an absorption characteristic for the rays of the part of the test subject based on the virtual system model.

6. The parameter optimization method as claimed in claim 1, wherein the examination component further comprises an examination table, the initial relative position information of the test subject relative to the examination component including initial relative position information of the test subject relative to the examination table.

7. The parameter optimization method as claimed in claim 1, wherein acquiring contour information of the test subject and the initial relative position information of the test subject relative to the examination component comprises:
acquiring an initial image of the test subject located in the medical device; and
determining contour information of the test subject and the initial relative position information thereof relative to the examination component based on the initial image.

8. The parameter optimization method as claimed in claim 1, further comprising:
causing the medical device to use the updated initial exposure parameter to perform exposure, so as to obtain a medical image;
determining an actual attenuation amount of the test subject based on an image parameter of the medical image; and
determining whether to again update the updated initial exposure parameter based on the estimated attenuation amount and the actual attenuation amount.

9. The parameter optimization method as claimed in claim 1, wherein the initial exposure parameter to be used by the medical device for performing exposure is determined according to a preset exposure curve describing an exposure parameter of each exposure point used by the medical device during operation, updating the initial exposure parameter based on the estimated attenuation amount including updating the initial exposure parameter according to the exposure curve based on the estimated attenuation amount.

10. A non-transitory computer-readable storage medium with an executable program stored thereon, that when executed, causes a processor to perform the method of claim 1.

11. A computer program product, embodied on a non-transitory computer-readable storage medium, and including a computer program that when executed by a processor, causes the processor to perform the method as claimed in claim 1.

12. A parameter optimization apparatus for a medical device, the medical device including an examination component having a radiation source and a detector for receiving rays emitted by the radiation source, and the parameter optimization apparatus comprising:
a first acquisition module adapted to acquire an initial exposure parameter usable by the medical device for performing exposure;
a second acquisition module adapted to acquire contour information of a test subject and initial relative position information of the test subject relative to the examination component;
a determining module adapted to determine an estimated attenuation amount of rays passing through the test subject at least based on the initial relative position information, contour information of the test subject, and real-time position information of the examination component, the estimated attenuation amount representing a degree of attenuation of rays from the radiation source after passing through the test subject, and the real-time position information of the examination component being position information of the examination component acquired in real time; and
an update module adapted to update the initial exposure parameter based on the estimated attenuation amount.

13. A medical device, comprising:
at least one processor; and
a memory in communicative connection with the at least one processor and storing a computer program which, when executed by the at least one processor, causes the at least one processor to:
acquire an initial exposure parameter usable by the medical device for performing exposure;
acquire contour information of a test subject and initial relative position information of the test subject relative to an examination component;
determine, based on the initial relative position information, the contour information, and real-time position information of the examination component, an estimated attenuation amount of rays passing through the test subject, he estimated attenuation amount representing a degree of attenuation of rays from a radiation source after passing through the test subject, and the real-time position information of an examination component being position information of the examination component acquired in real time; and
update the initial exposure parameter based on the estimated attenuation amount.
